(19) United States
(12) Patent Application Publication
(10) Pub. No.: US 2026/0346005 A1
(43) Pub. Date: Nov. 5, 2026

(54) MYOPIA OR PRESBYOPIA TRAINING DEVICE

(71) Applicants: Zhuhai ULook MetaBrain Medical Technology Co. Ltd., Zhuhai City (CN); METABRAIN TECHNOLOGY PTE.LTD., SINGAPORE (SG)

(72) Inventors: Wen-Fang LEI, Taoyuan City (TW); Shih-Hsien CHENG, Taoyuan City (TW); Shin-Da LEE, Taichung City (TW)

(21) Appl. No.: 19/194,506

(22) Filed: Apr. 30, 2025

(30) Foreign Application Priority Data
Oct. 10, 2024 (CN) 202411409031.2

(51) Int. Cl. A61N 1/36; A61N 1/04

(52) U.S. Cl. CPC A61N 1/36014; A61N 1/0472

(57) ABSTRACT

A myopia or presbyopia training device includes a bracket unit, a virtual display unit, a binaural audio stimulation unit, a transcranial stimulation unit and a control unit. The bracket unit includes top and back side parts, and two ears part. A virtual reality unit includes a display headset. The binaural audio stimulation unit includes paired speakers. The transcranial stimulation unit includes a first stimulus medium correspond to F3, F4, P1 and P2 positions in the international 10-20 brainwave electrode position, and the control unit is electrically connected with the display headset, the paired speakers and the first stimulus medium. Whereby, it can stimulate the user in ways in a single course of treatment through the display headset, the paired speakers and the first stimulus medium, achieving the interaction and synergistical effects of reducing intraocular pressure of the user and increasing visual acuity of the user.

### BACKGROUND OF THE INVENTION

### Field of the Invention

The invention relates to the technical field of myopia or presbyopia treatment, in particular to a myopia or presbyopia training device which can reduce intraocular pressure and increase visual acuity in users with myopia or presbyopia.

### Description of the Prior Art

The low visual acuity may be due to the blurred visual imaging caused by myopia and presbyopia. The main cause of myopia is usually caused by the elongation of the axial length. The intraocular pressure of normal people is 10-20 mmHg, while the intraocular pressure of patients with high myopia is mostly high, and high intraocular pressure will force the axial length to gradually lengthen. In addition, it is found that the visual system of patients with presbyopia will deteriorate as a whole, especially the nerves in the visual cortex. For the improvement of myopia or presbyopia, besides lens correction, medicine and surgery, how to reduce user's intraocular pressure and increase visual acuity through non-invasive approaches is also the focus of relevant research at present.

The present invention is, therefore, arisen to obviate or at least mitigate the above-mentioned disadvantages.

### SUMMARY OF THE INVENTION

The main object of the present invention is to provide a myopia or presbyopia training device which can provide various stimuli to the user in a single course of treatment through the display headset, the speaker and the first stimulus medium, thereby reducing the intraocular pressure of the user and increasing the visual acuity of the use.

To achieve the above and other objects, a myopia or presbyopia training device is provided, wherein the myopia or presbyopia training device includes: a bracket unit corresponding to the head shape of a user and comprising a top side part, a back side part and two ears part, wherein the two ears part respectively correspond to the ears of the user. The two ends of the top side part are respectively connected with the two ears part and straddle the top of the user's head upwards, and the two ends of the back side part are respectively connected with the two ears part and straddle the user's occipital bone backwards; a virtual reality unit comprising a display headset connected to the bracket unit and capable of switching between a display mode and a visual light flashing stimulation mode; wherein when the display headset is in the display mode, the display headset displays a virtual image, and the virtual image is used for guiding eyeball movement; when the display headset is in a visual light flashing stimulation mode, the display headset or a LED light displays light flashing with a specific wavelength and a specific frequency; the light flashing with the specific frequency and the specific wavelength is white or green light with a flashing rate of 40 Hz; a binaural audio stimulation unit comprising paired speakers, wherein the paired speakers are respectively connected to the two ears part and used for playing binaural beat music, and the binaural beat music has an binaural audio frequency difference wherein the binaural audio frequency difference of the binaural beat music is between 4 Hz and 18 Hz; a transcranial stimulation unit comprising a first stimulus medium, wherein the first stimulus medium is connected to the top side part to correspond to the F3 position, F4 position, P1 position and P2 position in the international 10-20 EEG electrode position; a control unit electrically connected with the display headset, the speaker and the first stimulus medium, wherein the control unit stores data information of the binaural beats music and the virtual image.

In some embodiments, the first stimulus medium is a first magnetic stimulation medium and is used for outputting electromagnetic pulses for transcranial magnetic stimulation; The first magnetic stimulation medium comprises an extension frame, a mounting seat and a magnetic coil, wherein the extension frame is connected to the support unit; The mounting seat has a first end and a second end which are opposite, the first end is connected to the extension frame, and the magnetic coil is connected to the mounting seat and electrically connected to the control unit.

Preferably, the mounting seat comprises a plurality of support layers stacked in sequence along the direction from the first end to the second end, and each support layer is provided with the magnetic coil; wherein, among any two adjacent support layers, the outer contour of one support layer close to the first end is larger than that of one support layer far from the first end, and the one support layer far from the first end is arranged in the middle of one support layer close to the first end.

More preferably, the end face of one end of the support layer far from the first end is a first magnetic surface, and a first area is surrounded by the magnetic coil around the center of the first magnetic surface on the first magnetic surface; Wherein, in any two adjacent support layers, one of the support layers far from the first end is arranged in the first area of one of the support layers near the first end.

Preferably, the control unit can control the magnetic coil to emit a plurality of current waves within a predetermined time T, each current wave has a rated current value, the rated current value of the current wave within the predetermined time is gradually increasing from small to large, and the rated current value is between 1 A and 10 A.

In some embodiments, the first stimulus medium is a first electrical stimulation medium, which comprises a connecting seat and a conductive column, the first electrical stimulation medium has a third end and a fourth end which are opposite, the third end is connected to the bracket unit, and the conductive column is connected to the fourth end and electrically connected to the control unit; The number of the conductive columns is multiple, and the conductive columns are distributed on the end face of the fourth end in parallel; The conductive column comprises a connecting part and a silicon electrode part, wherein the connecting part is provided with a fifth end and a sixth end which are opposite, the fifth end is connected to the fourth end, and the silicon electrode part is connected to the sixth end; The number of the silicon electrode parts are multiple, and the silicon electrode parts are distributed on the end face of the sixth end in parallel.

In some embodiments, the transcranial stimulation unit further comprises two second stimulus medium which are electrically connected with the control unit, and the two second stimulus medium are connected to the top side part to respectively correspond to the F7-T3 position, F8-T4 position of the user among the electrode positions of the International 10-20 EEG system: The second stimulus medium is used to output current to the F7-T3 position, F8-T4 position of the user for transcranial electrical stimulation or output electromagnetic pulse for transcranial magnetic stimulation.

In some embodiments, it also comprises an eye mask unit comprising an eye mask body, two eyes surrounding heating parts and eyes surrounding acupoint stimulation parts, The eye mask body is detachably connected to the bracket unit and spans the eyes of the user forward, and the eye mask body is provided with eye holes for exposing the eyes of the user and for watching virtual reality, The eyes surrounding heating parts are electrically connected with the control unit, and the eyes surrounding heating parts are connected with the eye mask body and used for thermally stimulating the periphery of the user's eyes through a first preset temperature; The eyes surrounding acupoint stimulation parts are electrically connected with the control unit, and ten eyes surrounding acupoint stimulation parts are connected with the eye mask body to correspond to the user's two Jingming BL1 acupoints, two Cuanzhu BL2 acupoints, two Yuyao EX-HN4 acupoints, two Sizhukong TE23 acupoints and two Chengqi ST1 acupoints respectively, and the eyes surrounding acupoint stimulation parts are used for respectively laser or thermal stimulating the two Jingming BL1 acupoints, two Cuanzhu BL2 acupoints, two Yuyao EX-HN4 acupoints, two Sizhukong TE23 acupoints and two Chengqi ST1 acupoints of the user through laser or a second preset temperature.

In some embodiments, it also comprises a head laser acupoint stimulation unit, wherein the head laser acupoint stimulation unit comprises three head laser acupoint stimulation medium and the head laser acupoint stimulation medium are electrically connected with the control unit and used for outputting laser, wherein the two head laser acupoint stimulation are connected to the back side part to respectively correspond to the two Fengchi GB20 acupoints of the user, another head laser acupoint stimulation medium is connected to the top side part to correspond to Baihui GV20 acupoint of the user, that is, three head laser acupoint stimulation mediums are used to respectively output laser light to the user's two Fengchi GB20 acupoints and one Baihui GV20 acupoint in the head for physical stimulation.

Preferably, the binaural audio frequency difference of the binaural beat music is 4 Hz-18 Hz, the laser wavelength of the head laser acupoint stimulation medium is 650-808 nm, and the light flashing at a specific frequency and wavelength is green light with a flashing rate of 40 Hz or 5 Hz-50 Hz.

In some embodiments, it also comprises an exercise bike unit comprising a bike frame, a flywheel and a pedal group, wherein the bike frame is used for the user to ride, The flywheel is rotatably connected to the bike frame, and the pedal group is in transmission connection with the flywheel and is used for being stepped by the user to rotate the flywheel around the central axis of the exercise bike.

Compared with the prior art, the myopia or presbyopia training device provided by the embodiment of the invention has the beneficial effects that: the display headset can be used for image training and illumination stimulation of the user's eyes, and binaural beat music with binaural audio frequency difference can be played to the user's ears through the speaker for music stimulation, the transcranial stimulation unit can output current to the F3 position, F4 position, P1 position and P2 position of the user's head for transcranial electrical stimulation or output electromagnetic pulse for transcranial magnetic stimulation, thereby reducing the intraocular pressure of the user and activating the visual center of the user; Therefore, the myopia or presbyopia training device provided by the invention can stimulate the user in a single course of treatment through the display headset, the paired speakers and the first stimulus medium, so as to achieve the synergistic effects of reducing the intraocular pressure of the user and increasing the visual acuity of the user.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a schematic structural diagram of a myopia or presbyopia training device provided by the first embodiment of the present invention;

FIG. 2 is a schematic diagram of the control connection structure of a myopia or presbyopia training device provided by the first embodiment of the present invention;

FIG. 3 is a partial structural schematic diagram of a myopia or presbyopia training device provided by the first embodiment of the present invention;

FIG. 4 is a side view of a myopia or presbyopia training device provided by the first embodiment of the present invention;

FIG. 5 is a front view of a myopia or presbyopia training device provided by the first embodiment of the present invention;

FIG. 6 is a schematic structural diagram of a first stimulus medium provided in embodiment 1 of the present invention;

FIG. 7 is a waveform diagram of the current applied by the magnetic coil of the control unit provided in the first embodiment of the present invention.

FIG. 8a-8e are display diagrams of five kinds of eye training exercises provided by the first embodiment of the present invention to reduce intraocular pressure and increase visual acuity, FIG. 8a illustrates the state that the pointing point moves upward or downward, FIG. 8b illustrates the state that the guide point moves to the upper right or lower left, FIG. 8c illustrates the state that the guide point moves to the left or right, FIG. 8d illustrates the state that the guide point moves to the lower right or upper left, and FIG. 8e illustrates the state that the guide point rotates clockwise or counterclockwise;

FIG. 9 is an experimental comparison chart of intraocular pressure values of ten myopia users provided by the embodiment of the present invention;

FIG. 10 is a comparison chart of myopia degree experiment of ten myopia users provided by the first embodiment of the present invention;

FIG. 11 is an experimental comparison chart of intraocular pressure values of ten presbyopia users provided by the first embodiment of the present invention;

FIG. 12 is a comparison chart of the presbyopia degree experiment of ten presbyopia users provided by the first embodiment of the present invention;

FIG. 13 is a diagram showing the effect relationship between multiple myopia training and increasing the percentage of visual acuity after training with a myopia or presbyopia training device provided by the first embodiment of the present invention; and

FIG. 14 is a schematic structural diagram of the first stimulus medium provided in the second embodiment of the present invention.

### DETAILED DESCRIPTION OF THE PREFERRED EMBODIMENT S

In the following, the specific embodiments of the present invention will be described in further detail with reference to the attached drawings and examples. The following examples are used to illustrate the invention, but are not used to limit the scope of the invention.

In the description of the present invention, it should be understood that the terms “center”, “vertical”, “horizontal”, “length”, “width”, “thickness”, “top” “bottom”, “front”, “back”, “left”, “right”, “vertical”, “horizontal”, “top”, “bottom” and “outside” etc. The indicated orientation or positional relationship is based on the orientation or positional relationship shown in the attached drawings. It is only for the convenience of describing the present invention and simplifying the description, and does not indicate or imply that the referred devices or elements must have a specific orientation, be constructed and operated in a specific orientation, so it cannot be understood as limiting the present invention.

The terms “first” and “second” are only used for descriptive purposes, and cannot be understood as indicating or implying relative importance or implicitly indicating the number of indicated technical features. Therefore, the features defined as “first” and “second” may include one or more of these features explicitly or implicitly.

In the present invention, unless otherwise specified and limited, the terms “connect” “connecting” “connection” should be broadly understood, for example, they can be fixed connection, detachable connection or integrated. It can be a mechanical connection or an electrical connection, can be directly connected, can also be indirectly connected through an intermediary, can be the internal connection of two elements or the interaction between two elements. For those skilled in the art, the specific meanings of the above terms in the present invention can be understood according to specific situations.

In the present invention, unless otherwise specified and limited, the first feature above or below the second feature may include direct contact between the first and second features, or may include that the first and second features are not in direct contact but through another feature contact between them. Moreover, the words “above”, “above” and “above” of the first feature include that the first feature is directly above and obliquely above the second feature, or only indicate that the horizontal height of the first feature is higher than the second feature. The words “under”, “under” and “under” of the first feature include that the first feature is directly below and obliquely below the second feature, or only indicate that the horizontal height of the first feature is smaller than the second feature.

### Embodiment 1

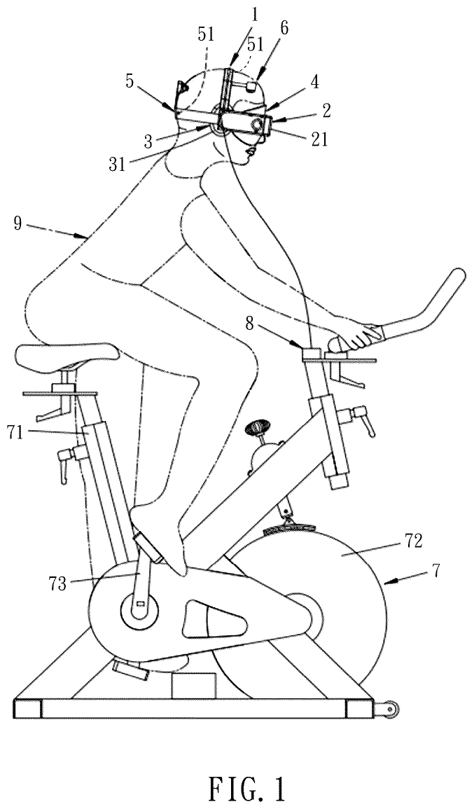

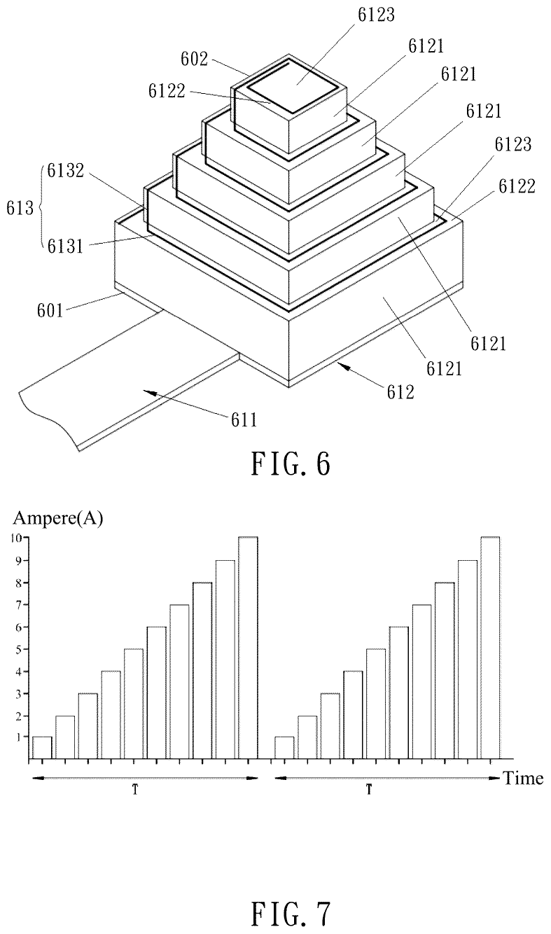

As shown in FIGS. 1-6, a myopia or presbyopia training device preferred by an embodiment of the present invention comprises a bracket unit 1, a virtual reality unit 2, a binaural audio stimulation unit 3, an eye mask unit 4, a head laser acupoint stimulation unit 5, a transcranial stimulation unit 6, an exercise bike unit 7 and a control unit 8.

Referring to FIGS. 1, 3, 4 and 5, the bracket unit 1 corresponds to the head shape of a user 9 and includes a top side part 12, a back side part 13 and two ears part 11, the two ears part 11 respectively correspond to the ears of the user 9, the two ends of the top side part 12 are respectively connected with the two ears 11 and straddle the top of the user 9 upwards, and the two ends of the back side part 13 are respectively connected with the two ears part 11 and straddle the occipital bone of the user 9 backwards.

Referring to FIGS. 1-5, the virtual reality unit 2 includes display headset 21 electrically connected to the control unit 8. The display headset 21 are connected to the bracket unit 1 and can switch between the display mode and the visual light flashing stimulation mode. When the display headset 21 are in the display mode, the display headset 21 display virtual images, which are used to guide eyes to see far and near and to guide eye movements. When the display headset 21 are in the visual light flashing stimulation mode, the display headset 21 or an extra LED light display flashing light with a specific wavelength and a specific frequency and stimulate the eyes of the user 9 by flashing light with a specific wavelength and a specific frequency. By using the display headset 21, the virtual image can be played through the display mode to guide the user's eyes to see far and near and guide the user's eyes to track and move, so as to achieve the better visual acuity via vision training. By using display headset 21, users' eyes can be stimulated by flashing at a specific wavelength and frequency through visual light flashing stimulation mode, so as to achieve lower intraocular pressure and increase visual acuity of users.

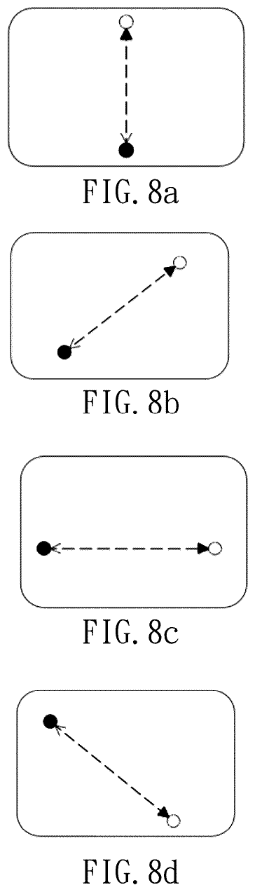

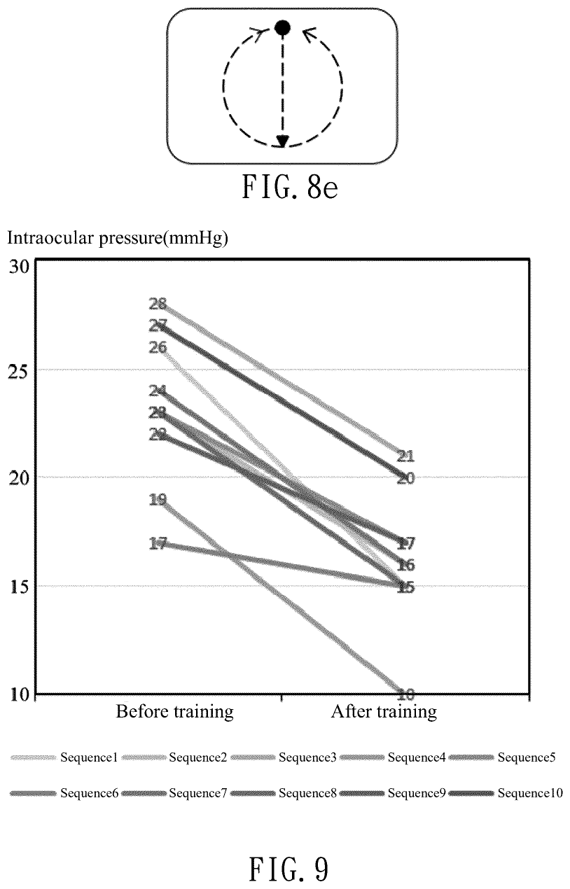

In this embodiment, the display headset 21 can display five virtual images of eye movement training as shown in FIGS. 8a-8e to guide the user to do eye movement. Of course, the display headset 21 can also display virtual images in any form, such as light spots and patterns, for the user to carry out eye movement training, but it is not limited thereto.

In this embodiment, the visual light flashing stimulation at a specific frequency and wavelength is green light with a flashing rate at 40 Hz or between 5 Hz-50 Hz. That is, when the display headset 21 are in the visual light flashing stimulation mode, the display headset 21 display green light with a flashing rate of 5 Hz-50 Hz.

The binaural audio stimulation unit 3 includes paired speakers 31, paired speakers 31 are connected to two ears part 11, respectively. Binaural beat music with a specific binaural audio frequency difference can be played through the speaker 31, so as to induce the user's brain waves to induce frequency following response and enter a specific brain wave form, activating the visual center to achieve better visual acuity in myopia or presbyopia via binaural beats music.

Preferably, the speaker 31 is used to (play binaural beats with frequency following response) to both ears of the user 9. The binaural beat music has a binaural audio frequency difference between 4 Hz and 18 Hz, which gradually decreases the binaural audio frequency difference from 18 Hz to 4 Hz.

Referring to FIGS. 1-5, the eye mask unit 4 includes an eye mask body 41, two eyes surrounding heating parts 42 and eyes surrounding acupoint stimulation parts 43, the eye mask body 41 is detachably connected to the bracket unit 1 and straddles the eyes of the user 9 forward, and the eye mask body 41 is provided with eye holes 411 for exposing the eyes of the user 9. The eyes surrounding heating parts 42 is electrically connected with the control unit 8, and the eyes surrounding heating parts 42 is connected with the eye mask body 41 and used for thermally stimulating the periphery of the eyes of the user 9 through a first preset temperature. The eyes surrounding acupoint stimulation parts 43 are electrically connected with the control unit 8, and the eyes surrounding acupoint stimulation parts 43 are connected with the eye mask body 41 and are used for respectively laser or thermal stimulating the two Jingming BL1 acupoints, two Cuanzhu BL2 acupoints, two Yuyao EX-HN4 acupoints, two Sizhukong TE23 acupoints and two Chengqi ST1 acupoints of the user 9 through a second preset temperature. Through the eye mask unit 4 when the user 9 performs eye training or visual light flashing stimulation through the display headset 21, at the same time, the eyes surrounding heating parts 42 and the eyes surrounding acupoint stimulation parts 43 are used to stimulate the periphery of the user's eyes via laser or thermal stimulation and specific eye acupoints, so as to stimulate the visual nervous activity, relax the eye muscles, and promote the blood circulation around the eyes, thus increase visual acuity via training device with eye mask unit 4.

In this embodiment, both ends of the eye mask body 41 are detachably connected to the two ears part 11.

In this embodiment, the first preset temperature is between 40° C. and 45° C.

In this embodiment, the second preset temperature of eyes surrounding acupoint stimulation parts are 50° C.

Preferably, the eyes surrounding heating parts 42 is made of graphene, and the eyes surrounding acupoint stimulation parts 43 is laser acupoint stimulation or made of graphene.

Referring to FIGS. 1-5, the head laser acupoint stimulation unit 5 includes three head laser acupoint stimulation medium 51, which are electrically connected with the control unit 8, wherein the two head laser acupoint stimulation medium 51 are connected to the back side part 13 and are respectively used for physically stimulating the two Fengchi GB20 acupoints of the user 9. Another head laser acupoint stimulation medium 51 is connected to the top side part and used to output laser light to a Baihui GV20 acupoint in the center of the user's head. Through the head laser acupoint stimulation medium 51, specific head laser acupoint stimulation can be stimulated by laser, so as to increase visual acuity via training device with head laser acupoint stimulation.

In this embodiment, the laser wavelength of the head laser acupoint stimulation medium 51 is 650-808 nm.

Referring to FIGS. 1-6, the transcranial stimulation unit 6 includes first stimulus mediums 61 and two second stimulus mediums 62. The first stimulus mediums are respectively connected to the top side part 12 to correspond to the F3 position, F4 position, P1 position and P2 position among the electrode positions of the International 10-20 EEG system, the first stimulus medium 61 is used to output current to the head of the user 9 for transcranial electrical stimulation or output electromagnetic pulse for transcranial magnetic stimulation. The first stimulus medium 61 is used to output current to the F3 position, F4 position, P1 position and P2 position of the user's head for transcranial electrical stimulation or output electromagnetic pulse for transcranial magnetic stimulation. Two second stimulus medium 62 are connected to the top side part 12 and are used to output current to the F7-T3 position, F8-T4 position of the user for transcranial electrical stimulation or output electromagnetic pulses for transcranial magnetic stimulation, respectively. The transcranial stimulation unit 6 can output current to the F3 position, F4 position, P1 position, P2 position and the F7-T3 position, F8-T4 position of the user's head for transcranial electrical stimulation or output electromagnetic pulses for transcranial magnetic stimulation.

In this embodiment, the first stimulus medium 61 is the first magnetic stimulation medium and is used to output electromagnetic pulses to the head of the user for transcranial magnetic stimulation. The first magnetic stimulation medium comprises an extension frame 611, a mounting seat 612 and a magnetic coil 613, wherein the extension frame 611 is connected to the bracket unit 1. The mounting seat 612 has a first end 601 and a second end 602 which are opposite to each other. The first end 601 is connected to the extension frame 611, and a magnetic coil 613 is arranged on the mounting seat 612. The magnetic coil 613 is electrically connected with the control unit 8 and can generate a magnetic force when power is applied.

In this embodiment, the extension frame 611 is connected to the top side part 12.

The mounting seat 612 includes a plurality of support layers 6121 stacked in sequence along the direction from the first end 601 to the second end 602, and each support layer 6121 is provided with a magnetic coil 613. Among any two adjacent support layers 6121, the outer contour of one support layer 6121 near the first end 601 is larger than that of one support layer 6121 far from the first end 601, and a support layer 6121 far from the first end 601 is arranged in the middle of a support layer 6121 near the first end 601. In this way, the mounting seat 612 can be made into a laminated tower structure that gradually narrows along the first end 601 to the second end 602, So that the support layer 6121 farthest from the first end 601 (that is, the support layer 6121 on the end of the second end 602) can be aligned with the F3 position, F4 position, P1 position and P2 position of the user, So that the magnetic force of the magnetic coil 613 can accurately stimulate the F3 position, F4 position, P1 position and P2 position of the user through the second end 602, which is helpful to reduce the intraocular pressure of the user and activate the visual center of the user to increase the visual acuity of the user.

In this embodiment, the number of support layers 6121 is five, and the five support layers 6121 are a first support layer, a second support layer, a third support layer, a fourth support layer and a fifth support layer which are sequentially distributed along the direction from the first end 601 to the second end 602, Therefore, the outer contour of the first supporting layer is larger than that of the second supporting layer, and the second supporting layer is arranged in the middle of the first supporting layer. The outer contour of the second supporting layer is larger than that of the third supporting layer, and the third supporting layer is arranged in the middle of the second supporting layer. The outer contour of the third supporting layer is larger than that of the fourth supporting layer, and the fourth supporting layer is arranged in the middle of the third supporting layer. The outer contour of the fourth support layer is larger than that of the fifth support layer, and the fifth support layer is arranged in the middle of the fourth support layer.

In this embodiment, the support layer 6121 has a square structure, and the length of the support layer 6121 with the largest outer contour is 3 cm, the width is 3 cm and the height is 1 cm. The supporting layer 6121 with the smallest outer contour is 1 cm in length, 1 cm in width and 1 cm in height. It can be adapted to the F3 position, F4 position, P1 position and P2 position of the head of the user 9, and effectively perform magnetic stimulation on the F3 position, F4 position, P1 position and P2 position of the head of the user 9, so as to further reduce the intraocular pressure of the user and increase the visual acuity of the user.

The end face of the support layer 6121 far from the first end 601 is a first magnetic surface 6122, and the magnetic coil 613 surrounds the center of the first magnetic surface 6122 on the first magnetic surface 6122 with a first area 6123, among any two adjacent support layers 6121, one support layer 6121 far from the first end 601 is arranged in the first area 6123 of one support layer 6121 near the first end 601. In this way, the magnetic coil 613 can be arranged in a spiral shape around a plurality of support layers 6121 to concentrate electromagnetic pulses. The first magnetic surface 6122 of a support layer 6121 (i.e., the fifth support layer) located on the second end 602 can be adapted to the P1 position or P2 position of the user. Therefore, the second end 602 can accurately stimulate the concentrated electromagnetic pulse to the F3 position, F4 position, P1 position and P2 position of the user, so as to further help reduce the intraocular pressure of the user and increase the visual acuity of the user.

Specifically, in any two adjacent support layers 6121, the magnetic coil 613 is arranged around the outer contour of the support layer 6121 far from the first end 601 on the first magnetic surface 6122 of the support layer 6121 near the first end 601, and extends from the first magnetic surface 6122 of the support layer 6121 near the first end 601 along the outer sidewall of the support layer 6121 far from the first end 601 to the first magnetic surface 6122 of the support layer 6121 far from the first end 601.

More specifically, the magnetic coil 613 includes a plurality of surrounding segments 6131 and a plurality of straight line segments 6132, a plurality of surrounding segments 6131 and straight line segments 6132 are alternately connected, and the surrounding segment 6131 is arranged on the first magnetic surface 6122 and encloses the first area 6123, and the straight line segment 6132 is arranged on the outer side wall of the support layer 6121, and the straight line segment 6132 is the shortest interconnection structure connecting the two first magnetic surfaces 6122, in this way, it can be adapted to the laminated tower structure of the mounting seat 612. The magnetic coil 613 can be made into a spiral-like structure that gradually narrows along the direction from the first end 601 to the second end 602 to concentrate the electromagnetic pulse along the direction from the first end 601 to the second end 602, it is also possible to have the shortest interconnection path between two adjacent surrounding segments 6131 to reduce the energy loss of the magnetic coil 613 to more effectively and stably convert electric power into magnetic force, therefore, the user's F3 position or F4 position can be more effectively magnetically stimulated, so as to further help reduce the user's intraocular pressure and increase the visual acuity of the user.

In this embodiment, the second stimulus medium 62 is a second magnetic stimulation medium, and the structure of the second magnetic stimulation medium is similar to that of the first magnetic stimulation medium, so further explanation is not given.

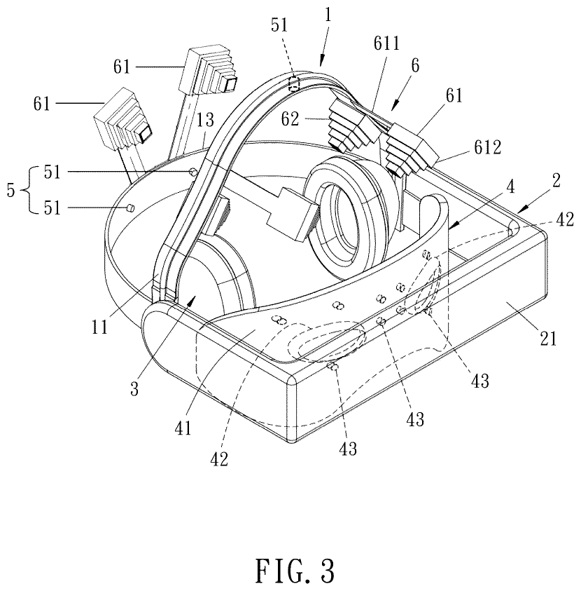

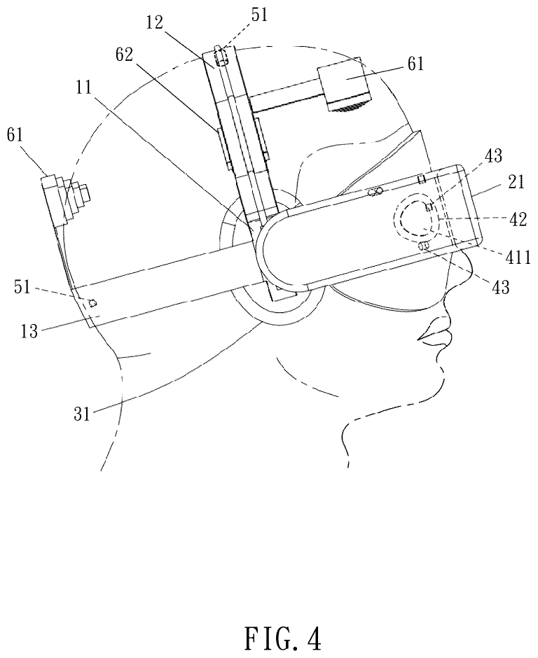

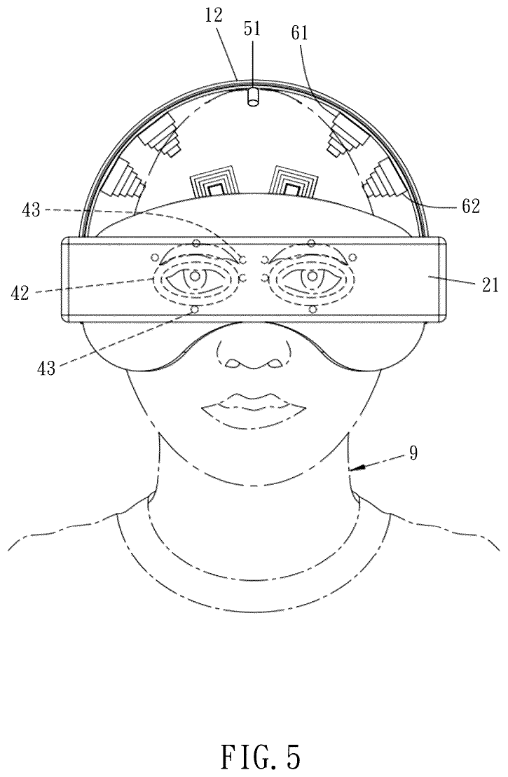

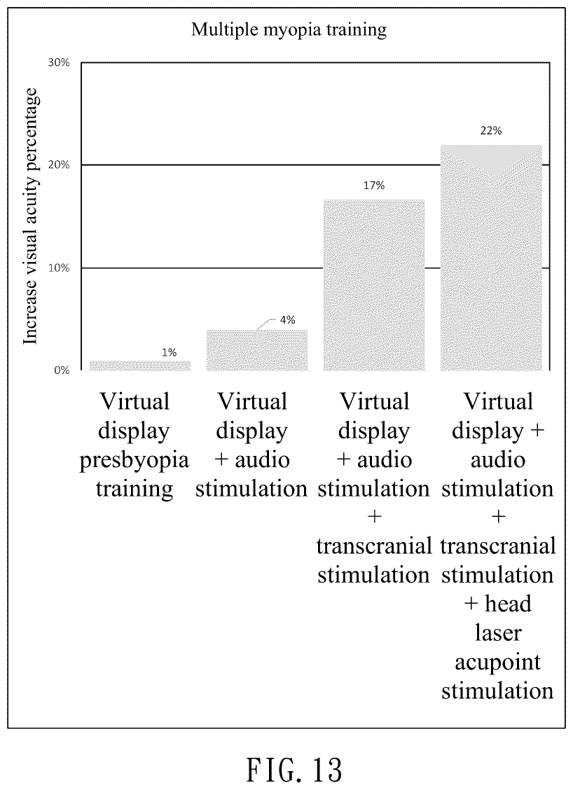

Referring to FIG. 1, FIG. 3, FIG. 4, and FIG. 5, in the present application, a virtual reality unit 2, a binaural audio stimulation unit 3, the eye mask unit 4, the head laser acupoint stimulation unit 5 and the transcranial stimulation unit 6 are all connected to the bracket unit 1, the bracket unit 1, the virtual reality unit 2, the binaural audio stimulation unit 3, the eye mask unit 4, the head laser acupoint stimulation unit 5 and the transcranial stimulation unit 6 can be formed into wearable training equipment. The wearable training equipment integrates a variety of training items, and users can choose any one or more trainings items according to their own needs, which is convenient for users to use. Referring to FIG. 13, the myopia or presbyopia training device provided by Embodiment 1 is used to train users for myopia in a single course of treatment, as can be seen from FIG. 13, the more myopia training items, the better the effect of increasing visual acuity, that is, a variety of myopia training items can achieve the synergistical effect of activating the user's visual center to increase the visual acuity of user with myopia.

Referring to FIG. 1, the exercise bike unit 7 includes a bike frame 71, a flywheel 72 and a pedal group 73, wherein the bike frame 71 is used for a user to ride, the flywheel 72 is rotatably connected to the bike frame 71, and the pedal group 73 is in transmission connection with the flywheel 72 and is used for a user 9 to step on to rotate the flywheel 72 around the central axis of the exercise bike 7.

In this embodiment, the exercise bike unit is electrically connected with the control unit 8. Specifically, the exercise bike unit 7 further includes a power module and an energy conversion module, the energy conversion module is respectively connected with the flywheel and the power module, and is used for converting the rotational potential energy of the flywheel into electric energy and storing the electric energy in the power module, and the power module is electrically connected with the control unit.

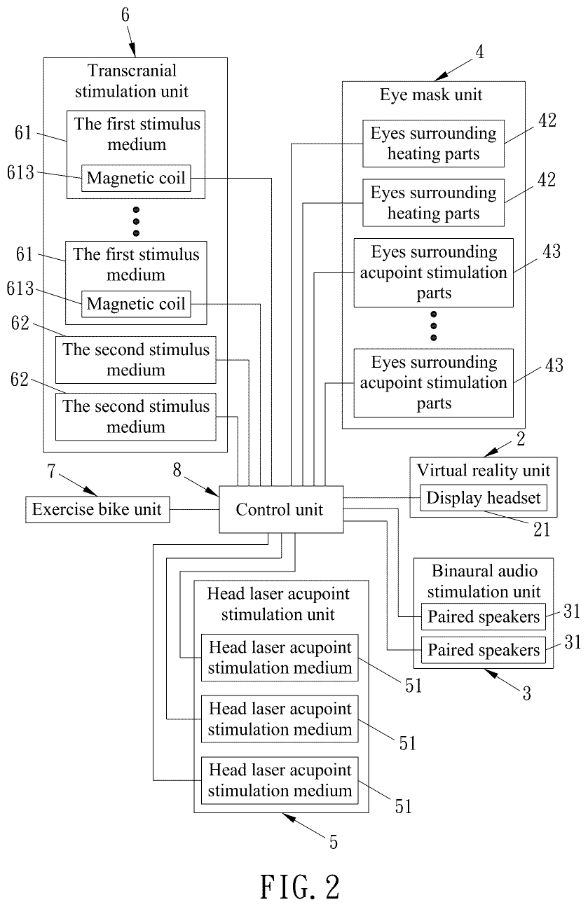

Referring to FIGS. 1 and 2, the control unit 8 is electrically connected with the display headset 21, the speaker 31, the eyes surrounding heating parts 42, eyes surrounding acupoint stimulation parts 43, head laser acupoint stimulation medium 51, a first stimulus medium 61, a second stimulus medium 62 and a power module 74. The control unit 8 stores data information of binaural beat music and virtual images, and the control unit 8 can also receive power from the power module, The control unit 8 can control the display headset 21 to display a virtual image or display flashing green light, and the control unit 8 controls the eyes surrounding heating parts 42 and the eyes surrounding acupoint stimulation parts 43 to heat, the control unit 8 controls the head laser acupoint stimulation medium 51 to emit laser light and controls the first magnetic stimulation medium and the second magnetic stimulation medium to emit magnetic stimulation.

In this embodiment, the control unit 8 is arranged on the exercise bike unit 7, but the control unit 8 can also be arranged on the virtual reality unit 2, and is not limited to this.

Referring to FIG. 7, in this embodiment, the control unit 8 can control the magnetic coil 613 to emit a plurality of current waves within a predetermined time T, each current wave has a rated current value, and the rated current value of the current waves within the predetermined time is gradually increasing from small to large, and the rated current value is between 1 A and 10 A.

In some embodiments, the predetermined time T is 1 second, and the number of current waves is 10 groups, but it can also be adjusted to 20 groups every 1 second as required, without limitation.

Referring to FIGS. 1-8e, the use method of the myopia or presbyopia training device provided by the embodiment of the present invention is as follows: Before use, the user 9 first wears the bracket unit 1, then sits on the bike frame 71 and steps on the pedal group 73 to prepare for the training of stepping on the flywheel. During training, the user 9 starts to step on the pedal group 73 for 30 minutes, at which time the control unit 8 controls the first stimulus medium 61 and the second stimulus medium 62 to emit magnetic stimulation to the user's head for 30 minutes, and at the same time, the head acupoint medium 51 is controlled to emit laser stimulation to the corresponding head acupoints for 30 minutes, and the eyes surrounding heating parts 42 and the eyes surrounding acupoint stimulation parts 43 are controlled to perform thermal therapy around the eyes and the corresponding eye acupoints for 30 minutes. In the first 15 minutes, the control unit 8 controls the display headset 21 to switch to the display mode, and controls the speaker 31 to play binaural beat music. In the next three minutes, the control unit 8 controls the display headset 21 to switch to the optical frequency stimulation mode, and controls the speaker 31 to stop playing binaural beat music and play health education content instead. In the last 12 minutes, the control unit 8 controls the display of the display headset 21 to switch to the display mode, and controls the speaker 31 to play the binaural beat music, thus completing the whole training.

It should be noted that when the display headset 21 are switched to the visual light flashing stimulation mode, the health education content played by the speaker 31 includes suggestions on diet, exercise, sleep, environmental arrangement, activity arrangement, etc. The following is a list of relevant contents, but it is not a restrictive condition.
I. Eat for healthy eyes. Nicotine is harmful to the retina, so you must quit smoking. Eat sweets in moderation and eat more colorful foods. Intake of wolfberry, blueberry, ginkgo leaf, etc. can protect the optic nerve. Appropriate intake of various vitamins and minerals such as calcium and iron can strengthen the sclera and slow down the increase in the length of the eye axis and the deepening of the degree.
II. Outdoor sports improve myopia and presbyopia. Outdoor activities for more than 2-3 hours a day. Looking at green plants more often helps eye health. The reason is that the green light band is most suitable for relieving eye fatigue, and the eyes can naturally relax. Therefore, going to see the distant mountain scenery and going to the mountains on holidays are good ways to care for vision.
III. Sleep well to improve myopia and presbyopia. Excessive use of eyes and lack of sleep are the main causes of myopia. Sleep well can enhance the development of the nervous system, and keep visual function well.
IV. Develop good eye habits. When reading, there should be sufficient light, and the light source should preferably come from the left rear. The reading posture should be correct, and the eyes should be 30-40 cm away from the book. Do not read while lying down or in a shaking car. The printing of books should be clear, and the font should not be too small or too crowded. Take a 10-minute break every 30 minutes of using your eyes (reading or using the computer), stand up and look into the distance, and do not overuse your eyes continuously.

When the display headset 21 are switched to the image mode, the display image shows the eye training movement. Referring to FIGS. 8a-8e, in this embodiment, the display image is presented in the way that the guiding point moves in the picture, So that the user 9 can change the position of the eyeball with the pointing point, and move upward or downward (see FIG. 8a), upward or downward (see FIG. 8b), Move to the left or right (see FIG. 8c), move to the lower right or move to the upper left (see FIG. 8d), Turn clockwise or counterclockwise (see FIG. 8e) and change the distance with the three-dimensional virtual image to change the focal length of the user's eyes.

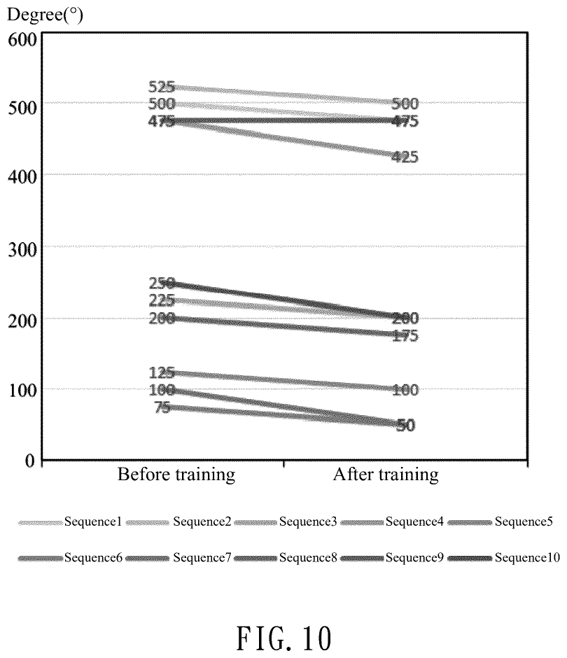

Referring to FIGS. 9-10, the experiment was conducted with ten myopic users with intraocular pressure ranging from 17 mmHg to 28 mmHg, and the series 1-10 respectively correspond to the ten myopic users, ten myopic users obtained intraocular pressure values through tonometer detection and myopia degrees through optometry detection, wherein each user uses the myopia or presbyopia training device provided by this embodiment once for 30 minutes. The use mode is to listen to the binaural beat music and stimulate the F3 position, F4 position, P1 position, P2 position and the F7-T3 position, F8-T4 position of the user, the intraocular pressure value before training in FIG. 9 is the intraocular pressure value measured by the user before using the myopia or presbyopia training device provided by this embodiment, the intraocular pressure value after training in FIG. 9 is the intraocular pressure value measured by the user after using the myopia or presbyopia training device provided by this embodiment, the pre-training degree in FIG. 10 refers to the myopia degree measured by the user before using the myopia or presbyopia training device provided by this embodiment, and the post-training degree in FIG. 10 refers to the myopia degree measured by the user after using the myopia or presbyopia training device provided by this embodiment.

As can be seen from FIGS. 9-10, 10 myopic users with high original intraocular pressure between 17 mmHg and 28 mmHg listened to binaural beat music and stimulated F3 position, F4 position, P1 position, P2 position and the F7-T3 position, F8-T4 position with the myopia or presbyopia training device provided by the embodiment of the present invention, after training, the intraocular pressure value decreased by 7 mmHg on average, and the myopia degree decreased by 30 degrees on average.

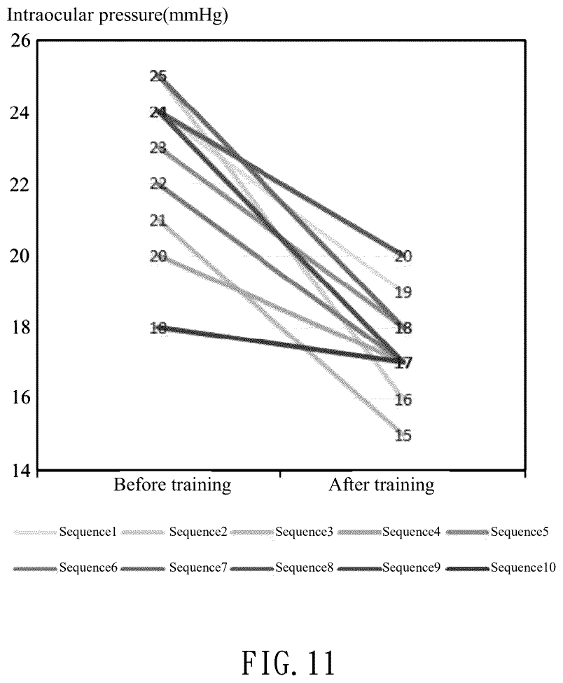

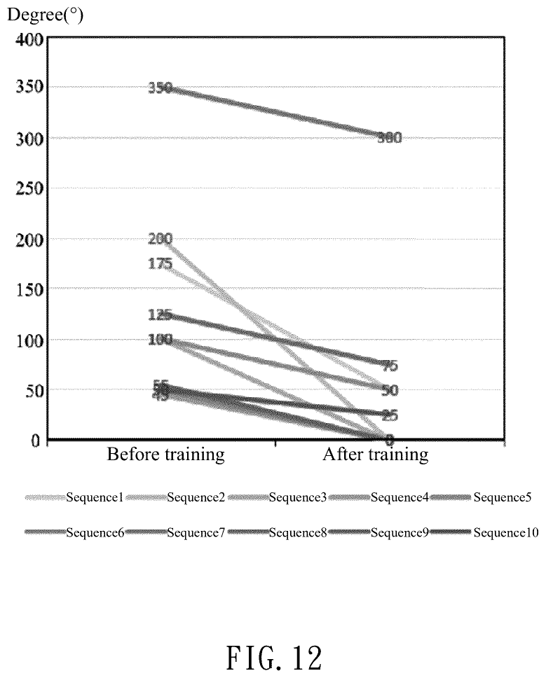

Referring to FIGS. 11-12, the experiment was carried out with ten presbyopia users whose intraocular pressure ranged from 18 mmHg to 25 mmHg, the series 1-10 respectively correspond to ten presbyopia users, and the intraocular pressure values obtained by the ten presbyopia users are detected by the tonometer and the presbyopia degrees are obtained by the optometry instrument, wherein each user uses the myopia or presbyopia training device provided by this embodiment once for 30 minutes. The use mode is to listen to the binaural beat music and stimulate the F3 position, F4 position, P1 position, P2 position and the F7-T3 position, F8-T4 position of the user, The intraocular pressure value before training in FIG. 11 is the intraocular pressure value measured by the user before using the myopia or presbyopia training device provided by this embodiment. The intraocular pressure value after training in FIG. 11 is the intraocular pressure value measured by the user after using the myopia or presbyopia training device provided by this embodiment. The pre-training degree in FIG. 12 refers to the presbyopia degree measured by the user before using the myopia or presbyopia training device provided by this embodiment. The post-training degree in FIG. 12 refers to the presbyopia degree measured by the user after using the myopia or presbyopia training device provided by this embodiment.

As can be seen from FIGS. 11-12, ten presbyopic users who have high original intraocular pressure values between 18 mmHg and 25 mmHg are listening to the binaural beat music by using the myopia or presbyopia training device provided by the embodiment of the invention, after stimulation at F4 position, P1 position, P2 position and the F7-T3 position, F8-T4 position, the average intraocular pressure after training decreased by 5.2 mmHg, and the average presbyopia decreased by 75 degrees.

From FIG. 9 to FIG. 12, it can be proved that listening to binaural beat music, stimulating the F7-T3 position, F8-T4 position of the user's head and stimulating the brain regions at F3 position, F4 position, P1 position and P2 position can reduce the user's intraocular pressure and increase the user's visual acuity.

Referring to FIGS. 1 and 3, the improvement of intraocular pressure caused by the laser or thermal stimulation of the corresponding eyes surrounding acupoints by the eyes surrounding acupoint stimulation parts 43 and the stimulation of the corresponding head acupoint by the head laser acupoint stimulation medium 51 can be proved by the following references, so this description will not be further described.
1. Takayama, S., et al., Short-term effects of acupuncture on open-angle glaucoma in retrobulbar circulation: additional therapy to standard medication. Evid Based Complement Alternat Med, 2011. 2011: p. 157090.
2. Liu, W., et al., Impact of acupuncture on 24 h intraocular pressure of glaucoma. Zhongguo zhen jiu=Chinese acupuncture & moxibustion, 2011. 31(6): p. 518-520.
3. Yeh, T. Y., J. C. Lin, and C. F. Liu, Effect of transcutaneous electrical nerve stimulation through acupoints of Pucan (BL 61) and Shenmai (BL 62) on intraocular pressure in patients with glaucoma: a randomized controlled trial. J Tradit Chin Med, 2016. 36(1): p. 51-6.
4. Chen, J., et al., Acupuncture Treatment Reverses Retinal Gene Expression Induced by Optic Nerve Injury via RNA Sequencing Analysis. Front Integr Neurosci, 2 019. 13: p. 59.

In addition, exercise and visual green light stimulation can reduce intraocular pressure and increase visual acuity, which can be proved by the following references, so this specification will not be further explained.
5. Meier, N. F., et al., Physical Activity, Cardiorespiratory Fitness, and Incident Glaucoma. Med Sci Sports Exerc, 2018. 50 (11): p. 2253-2258.
6. Sergienko, N. M. and I. V. Shargorodskaia, [Effects of monochromatic light on ocular hydrodynamics]. Vestn Of talmol, 2000. 116(4): p. 17-9.

To sum up, the myopia or presbyopia training device provided by the embodiment of the invention is provided with a virtual reality unit 2, a paired speakers 31 for playing binaural beat music, an head laser acupoint stimulation unit 5 for providing acupoint stimulation, a transcranial stimulation unit 6 for providing magnetic stimulation, the eye mask unit 4 for providing thermal stimulation and the exercise bike unit 7 for training can provide users with various stimuli in a single course of treatment, so as to achieve the synergistical effects of reducing the intraocular pressure of users and increasing the visual acuity.

### Embodiment 2

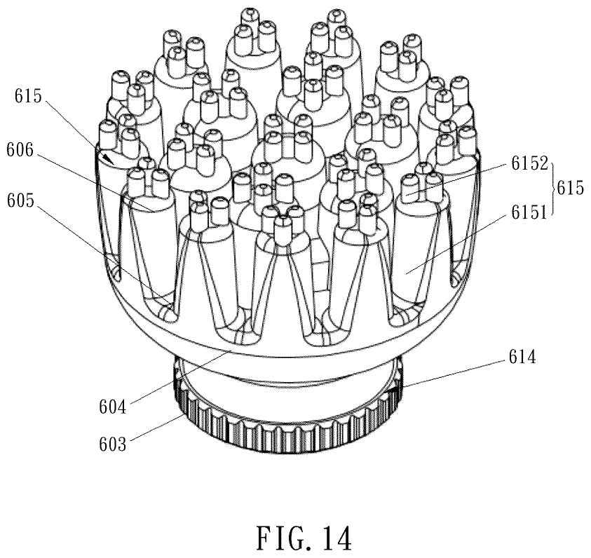

Different from the first embodiment, referring to FIG. 14, the first stimulus medium 61 provided by the second embodiment is the first electrical stimulation medium.

The first stimulus medium 61 is the first electrical stimulation medium and is used to output current to the F3 position, F4 position, P1 position and P2 position of the user's head for transcranial electrical stimulation. The first electrical stimulation medium is in the form of direct or alternating current output, and the current of the direct current or alternating current is between 0.01-3 mA.

In this embodiment, the first electrical stimulation medium includes a connecting seat 614 and a conductive column 615. The connecting seat 61 has a third end 603 and a fourth end 604 opposite to each other, the third end 603 is connected to the bracket unit 1, and the conductive column 615 is connected to the fourth end 604 and electrically connected to the control unit 8.

The number of conductive columns 615 is multiple, and the plurality of conductive columns 615 are distributed in parallel on the end face of the fourth end 604 and are used for providing 0.01-3.0 mA of direct current or alternating current. A plurality of conductive columns 615 are connected to the end face of the connecting seat 614, so that a single conductive column 615 can have a smaller volume, so that the single conductive column 615 and the user 9 have a smaller single contact area, and the smaller single contact area and many contact areas can reduce the current density through the skin, which is helpful to reduce the potential stimulation risk or discomfort. At the same time, the plurality of conductive columns 615 can form a plurality of contact positions with the user 9 and form a larger coverage area, thereby ensuring that the first electrical stimulation medium can cover the F3 position or F4 position or P1 position or P2 position of the user 9.

In this embodiment, the end face of the fourth end 604 has a circular structure, and the center of the end face of the fourth end 604 is provided with a conductive column 615. The end face of the fourth end 604 has a plurality of arrangement areas distributed at intervals along its radial direction, and each arrangement area has a plurality of conductive columns 615 distributed around the center of the end face of the fourth end 604. In this way, a plurality of conductive columns 615 can be densely arranged on the end face of the circular fourth end 604 to form a circular coverage area, so that the first electrical stimulation medium can completely cover the area to be stimulated of the user 9 and make part of the conductive columns 615 accurately align with the F3 position or F4 position or P1 position or P2 position of the user 9, Further, the current of the conductive column 615 can accurately stimulate the F3 position or F4 position or P1 position or P2 position of the user, and the conduction can provide 0.01-3.0 mA of direct current or alternating current, so as to effectively reduce the intraocular pressure of the user and increase the visual acuity of the user.

Specifically, in this embodiment, the number of arrangement regions is two.

In other embodiments, the number of arrangement regions can also be any number such as one, three or four, which is not limited here.

The conductive column 615 comprises a connecting part 6151 and a silicon electrode part 6152, wherein the connecting part 6151 has a fifth end 605 and a sixth end 606, the fifth end 605 is connected to the fourth end 604, and the silicon electrode part 6152 is connected to the sixth end 606. The number of silicon electrode portions 6152 is plural, and the plurality of silicon electrode portions 6152 are distributed in parallel on the end face of the sixth end 606. The adoption of a plurality of silicon electrode parts 6152 enables a single silicon electrode part 6152 to have a smaller volume to pass through the hair, so that the single silicon electrode part 6152 has a smaller single contact area and many contact areas with the user 9, and the smaller single contact area and many contact areas can reduce the current density through the skin, and the intensity of current is extremely small without stimulation risk or discomfort. At the same time, because the material of the silicon electrode part 6152 is soft, the silicon electrode part 6152 can be in soft contact with the user 9, which is helpful to further reduce the potential stimulation risk or discomfort.

The silicon electrode part 6152 is made of silicon electrode material.

The material of the connecting part 6151 and the material of the silicon electrode part 6152 may be the same or different.

The above is only the preferred embodiment of the present invention, it should be pointed out that a person skilled in the art can make several improvements and substitutions without departing from the technical principles of the present invention, and these improvements and substitutions should also be regarded as the protection scope of the present invention.

What is claimed is:

1. A myopia or presbyopia training device including:
a bracket unit corresponding to the head shape of a user and comprising a top side part, a back side part and two ears part, wherein the two ears part respectively correspond to the ears of the user, The two ends of the top side part are respectively connected with the two ears part and straddle the top of the user's head upwards, and the two ends of the back side part are respectively connected with the two ears part and straddle the user's occipital bone backwards;
a virtual reality unit comprising a display headset connected to the bracket unit and capable of switching between a display mode and a visual light flashing stimulation mode; wherein when the display headset is in the display mode, the display headset displays a virtual image, and the virtual image is used for guiding eyeball movement; when the display headset is in a visual light flashing stimulation mode, the display headset or a LED light displays light flashing with a specific wavelength and a specific frequency; the light flashing with the specific frequency and the specific wavelength is white or green light with a flashing rate of 40 Hz;
a binaural audio stimulation unit comprising paired speakers, wherein the paired speakers are respectively connected to the two ears part and used for playing binaural beat music, and the binaural beat music has an binaural audio frequency difference wherein the binaural audio frequency difference of the binaural beat music is between 4 Hz and 18 Hz;
a transcranial stimulation unit comprising a first stimulus medium, wherein the first stimulus medium is connected to the top side part to correspond to the F3 position, F4 position, P1 position and P2 position in the international 10-20 EEG electrode position;
a control unit electrically connected with the display headset, the speaker and the first stimulus medium, wherein the control unit stores data information of the binaural beats music and the virtual image.

2. The myopia or presbyopia training device according to claim 1, wherein the first stimulus medium is a first magnetic stimulation medium, the first magnetic stimulation medium comprises an extension frame, a mounting seat and a magnetic coil, the extension frame is connected to the top side part, the mounting seat has a first end and a second end which are opposite, the first end is connected to the extension frame, and the magnetic coil is connected to the mounting seat and electrically connected to the control unit.

3. The myopia or presbyopia training device according to claim 2, wherein the mounting seat comprises a plurality of support layers stacked in sequence along the direction from the first end to the second end, and each support layer is provided with the magnetic coil; among any two adjacent support layers, the outer contour of one support layer close to the first end is larger than that of one support layer far from the first end, and the one support layer far from the first end is arranged in the middle of one support layer close to the first end.

4. The myopia or presbyopia training device according to claim 3, wherein an end face of the support layer far from the first end is a first magnetic surface, and the magnetic coil encloses a first area around the center of the first magnetic surface on the first magnetic surface; among any two adjacent support layers, one support layer far from the first end is arranged in the first area of one support layer close to the first end.

5. The myopia or presbyopia training device according to claim 2, wherein the control unit can control the magnetic coil to emit a plurality of current waves within a predetermined time T, each current wave has a rated current value, and the rated current values of the current waves within the predetermined time are gradually increasing from small to large, and the rated current values are between 1 A and 10 A.

6. The myopia or presbyopia training device according to claim 1, wherein the first stimulus medium is a first electrical stimulation medium, the first electrical stimulation medium comprises a connecting seat and a conductive column, the first electrical stimulation medium is provided with a third end and a fourth end, the third end is connected to the bracket unit, and the conductive column is connected to the fourth end and electrically connected to the control unit; the number of the conductive columns is multiple, and the conductive columns are distributed on the end face of the fourth end in parallel and used for providing 0.01-3.0 mA of direct current or alternating current; the conductive column comprises a connecting part and a silicon electrode part, wherein the connecting part is provided with a fifth end and a sixth end which are opposite, the fifth end is connected to the fourth end, and the silicon electrode part is connected to the sixth end; the number of the silicon electrode parts are multiple, and the silicon electrode parts are distributed on the end face of the sixth end in parallel.

7. The myopia or presbyopia training device according to claim 1, wherein the transcranial stimulation unit further comprises two second stimulus medium, the second stimulus medium are electrically connected with the control unit, and two second stimulus medium are connected to the top side part to respectively correspond to the F7-T3 position, F8-T4 position of the user among the electrode positions of the International 10-20 EEG system.

8. The myopia or presbyopia training device according to claim 1, wherein the myopia or presbyopia training device further comprises an eye mask unit and an head laser acupoint stimulation unit; the eye mask unit comprises an eye mask body, two eyes surrounding heating parts and ten eyes surrounding acupoint stimulation parts, wherein the eye mask body is detachably connected to the bracket unit and straddles the eyes of the user forward, the eye mask body is provided with eye holes for the eyes of the user to be exposed, the eyes surrounding heating parts are electrically connected with the control unit, and the eyes surrounding heating parts are connected with the eye mask body; the eyes surrounding acupoint stimulation parts are electrically connected with the control unit, and ten eyes surrounding acupoint stimulation parts are connected with the eye mask body and correspond to the user's two Jingming BL1 acupoints, two Cuanzhu BL2 acupoints, two Yuyao EX-HN4 acupoints, two Sizhukong TE23 acupoints and two Chengqi ST1 acupoints respectively.

9. The myopia or presbyopia training device according to claim 1, wherein the myopia or presbyopia training device further comprises a head laser acupoint stimulation unit comprises three head laser acupoint stimulation medium, which are electrically connected with the control unit and used for outputting heat or laser, wherein two head laser acupoint stimulation medium are connected to the back side part to correspond to the user's two Fengchi GB20 acupoints respectively, and the other head laser acupoint stimulation medium is connected to the top side part to correspond to the user's Baihui GV20 acupoint, and the laser wavelength of the eyes surrounding acupoint stimulation parts and the head laser acupoint stimulation medium is 650-808 nm.

10. The myopia or presbyopia training device according to claim 1, wherein the myopia or presbyopia training device further comprises an exercise bike unit comprising a bike frame, a flywheel and a pedal group, the bike frame is used for the user to ride, the flywheel is rotatably connected to the bike frame, the pedal group is in transmission connection with the flywheel and is used for the user to step on to make the flywheel rotate around the central axis of the flywheel, the speed and distance of exercise bike can transmit to the control unit and further link and display those to the virtual reality unit, and the exercise bike unit can store the electric energy and provide electric energy supply to the myopia or presbyopia training device.
